(19) United States
(12) Patent Application Publication
(10) Pub. No.: US 2023/0329608 A1
(43) Pub. Date: Oct. 19, 2023

(54) METHOD AND SYSTEM FOR ESTIMATING SYMPATHETIC AROUSAL OF A SUBJECT

(71) Applicant: Philia Labs Pty Ltd, New South Wales (AU)

(72) Inventors: Dilpreet Singh Buxi, Noble Park (AU); Alexander Paul Senior, Black Rock (AU); Katayoon SARAFRAZI, PointCook (AU); David Lester, South Yarra (AU); Vinayak Smith MM, Wheelers Hill (AU)

(73) Assignee: Philia Labs Pty Ltd, New South Wales (AU)

(21) Appl. No.: 18/022,510

(22) PCT Filed: Aug. 20, 2021
(86) PCT No.: PCT/AU2021/050920

§ 371 Date: Feb. 21, 2023

(30) Foreign Application Priority Data
Aug. 21, 2020 (AU) 2020902998

(51) Int. Cl. A61B 5/0533; A61B 5/024; A61B 5/16; A61B 5/00

(52) U.S. Cl. CPC A61B 5/165; A61B 5/02405; A61B 5/0533; A61B 5/4035; A61B 5/7246

(57) ABSTRACT

A method (100) for estimating sympathetic arousal of a subject, the method (100) comprising the steps of: obtaining physiological data (500) from the subject (102), the physiological data (500) including at least one of galvanic skin response data, skin blood perfusion data, and heart rate data of the subject; processing the physiological data (500) to determine one or more features of the physiological data (500); providing the one or more features of the physiological data (500) to a correlation engine (300), the correlation engine (300) configured to correlate the one or more features of the physiological data (500) with a database (400) representing sympathetic nervous activity signals to estimate a sympathetic nervous activity level of the subject; and generating an output of the estimated sympathetic nervous activity level of the subject.

### CROSS REFERENCE TO RELATED APPLICATION

The present application claims priority to Australian Provisional Patent Application No 2020902998, filed on 21 Aug. 2020, the content of which is incorporated herein by reference in its entirety.

### TECHNICAL FIELD

The present disclosure is directed to a method and a system for estimating sympathetic arousal of a subject and, therefore, stress levels of the subject.

### BACKGROUND

Many aspects of stress in mammals represent positive physiological phenomena, when manifested in the right situations, in the right amount and in the right balance. Under stress conditions, the sympathetic nervous system mobilises the resources in the individual to handle a situation of imminent danger in the optimum manner. The result is that the individual’s mental processes and key senses are sharpened, while less relevant sensory impressions are impeded. Physically, the body responds by lowering response times, increasing muscle strength and optimising motor skills coordination. Physiologically, simulation via the sympathetic nerve system increases the pulse and the blood pressure and inhibits secretion formation in the glands. This response activation affects all the systems of the body, including the brain, the cardiovascular system, the respiratory system, the immune system and the digestive system. Once the challenge of the immediate situation has passed, the body’s natural response is inactivated and the body moves to a recovery phase.

However, if the situation represents a state in which the strain surpasses the body’s resources, those resources become taxed and long-term stress will result, leading to medical impairment. Stress reaction is not activated only by purely physical or psychological threats, but also through mental and environmental condition. Personal worries in situations of everyday life, particularly accumulating over extended periods, can result in activation the response phase without any clear immediate threatened, also leading to negative or chronic stress.

Chronic stress may thus have a significant negative impact on health. In mild forms, this can manifest as fatigue, muscle tension or fatigue, while at a more severe level symptoms can include memory problems, lack of concentration, sleep disorders, hypertension, anxiety, depression, chronic fatigue, digestion problems, palpitations, decreased libido, asthma, and muscular diseases. Untreated chronic stress may lead to various illnesses, some of which can be fatal. Overall, stress-related problems come at a huge cost to economies around the world, in the region of billions of dollars annually.

In managing or avoiding development of stress responses, determination of an individual’s stress level is important. This allows suitable actions to be initiated in order to reduce or otherwise address the causal factors or the individual’s ability to handle these factors strains can be increased such that the negative consequences can be averted.

There are several know methods for determining an individual’s stress level (ie. the activity of the sympathetic nervous system). One known method is measuring the individual’s cortisol levels. This method, however, requires the subject to attend a medical facility to have their cortisol levels tested. Further, laboratory analysis is required, meaning that such tests can be relatively expensive and there can be a significant delay in receiving the results.

Another method for assessing an individual’s stress level is through the use of a questionnaire filled out by the individual. This approach does not typically produce reliable results as it is highly subjective, the usefulness depending on the individual’s truthful answers to the questions posed.

Other methods for recording and managing an individual’s stress include measuring heart rate, breathing rate, and blood pressure, e.g. by way of a wearable device worn by the individual. Although these methods may be realised in the ambulatory environment, the data obtained from such wearable devices is typically subject to post-processing methods that can reduce the correlation between the data and the person’s sympathetic arousal, meaning that any appraisal of a subject’s stress levels derived from this data is also inaccurate and consequently may not be attributable solely to the stress response.

There thus exists a need for a fast, reliable and inexpensive method of determining a subject’s stress level.

Reference to any prior art in the specification is not an acknowledgement or suggestion that this prior art forms part of the common general knowledge in any jurisdiction or that this prior art could reasonably be expected to be combined with any other piece of prior art by a skilled person in the art.

### SUMMARY

In a first aspect, the present invention provides a method for estimating sympathetic arousal of a subject, the method comprising the steps of:
obtaining physiological data from the subject, the physiological data including at least one of galvanic skin response data, skin blood perfusion data, and heart rate data of the subject;
processing the physiological data to determine one or more features of the physiological data;
providing the one or more features of the physiological data to a correlation engine, the correlation engine configured to correlate the one or more features of the physiological data with a database representing sympathetic nervous activity signals to estimate a sympathetic nervous activity level of the subject; and
generating an output of the estimated sympathetic nervous activity level of the subject.

In a second aspect, the present invention provides a method of developing a correlation engine for estimating sympathetic arousal of a subject, the method comprising the steps of:
generating a database of sympathetic nervous activity signals by recording and processing sympathetic nervous activity signals from a plurality of participants;
obtaining physiological data from each participant, the physiological data of each participant including at least one of galvanic skin response data, skin blood perfusion data, and heart rate data of the subject;
processing the physiological data obtained from each participant to calculate one or more features of the physiological data; and
correlating the sympathetic nervous activity signals of each participant with the one or more features of the physiological data obtained from the respective participant to develop the correlation engine,
wherein the correlation engine is configured to be used for correlating one or more features of physiological data obtained from the subject with representations of the sympathetic nervous activity signals in order to output an estimated sympathetic nervous activity level of the subject.

In a preferred form, the representations of sympathetic nervous activity may be obtained by recording one or more sympathetic nervous activity signals and/or one or more impedance cardiography (ICG) signals from multiple participants at different stress levels.

In a third aspect, the present invention provides a system of estimating sympathetic arousal of a subject, the system comprising:
a device configured to obtain physiological data of the subject, the physiological data including at least one of galvanic skin response data, photoplethysmography data, and heart rate data of the subject;
a processor to process the physiological data to calculate one or more features of the physiological data; and
a correlation engine configured to correlate the one or more features of the physiological data with a database representing sympathetic nervous activity signals to estimate a sympathetic nervous activity level of the subject and output an estimate of a sympathetic nervous activity level of the subject.

Preferably, in accordance with the above-defined aspects of the invention, the physiological data obtained and processed includes measures of galvanic skin response data, skin blood perfusion data, and heart rate data from the subject or participant.

In another broad form, the invention provides a method for estimating sympathetic arousal of a subject, comprising obtaining physiological data from the subject, the physiological data including at least one of galvanic skin response data, skin blood perfusion data, and heart rate data of the subject, processing the physiological data to determine one or more features thereof and provide a physiological data pattern, providing the physiological data pattern to a correlation engine, the correlation engine configured to map physiological data patterns against stored measures of sympathetic nervous activity which have been built up from empirical observations of correlation between sympathetic nervous activity signals and physiological data signals, and outputting from the correlation engine an sympathetic nervous activity score for the subject.

The present invention therefore makes use of a knowledge base built up from intraneural recordings of sympathetic nerve activity from multiple subjects. This allows a prediction in changes in sympathetic nerve activity by processing physiological signals, allowing a quantification of stress in subjects, accurately and in real time.

### BRIEF DESCRIPTION OF THE DRAWINGS

Preferred embodiments of the invention are described, by way of examples only, with reference to the accompanying figures, wherein:

FIG. 1 is a flowchart of a method for estimating a stress level of a subject according to an embodiment of the present invention;

FIG. 2 is a method of generating a correlation engine for estimating a stress level of a subject and generating a database of sympathetic nervous activity recordings according to an embodiment of the present invention for use in the method of FIG. 1;

FIG. 3A is a raw sympathetic nervous activity recording obtained from one of a plurality of participants for use in generating the correlation engine and database of FIG. 2;

FIG. 3B shows an RMS sympathetic nervous activity signal produced by processing the raw sympathetic nervous activity recording of FIG. 3A;

FIG. 4 is flow diagram illustrating an example method of processing a raw sympathetic nervous activity signal to remove noise from the raw sympathetic nervous activity signal;

FIG. 5 is a graph illustrating the estimated skin sympathetic nervous activity level for a given number of sympathetic nervous activity bursts per minute calculated from the sympathetic nervous activity recordings obtained from the plurality of participants;.

FIGS. 6A and 6B are examples of galvanic skin response data, skin blood perfusion data, and heart rate data;

FIG. 7 is an example of a sigmoidal function;

FIG. 8 illustrates the inputs and output of the correlation engine generated in FIG. 2;

FIG. 9 is another a method of generating a correlation engine for estimating a stress level of a subject and generating a database of sympathetic nervous activity recordings according to an embodiment of the present invention for use in the method of FIG. 1;

FIG. 10 is an example of an impedance cardiography (ICG) signal and electrocardiogram (ECG) signal obtained from a participant;

FIG. 11 is another a method of generating a correlation engine for estimating a stress level of a subject and generating a database of sympathetic nervous activity recordings according to an embodiment of the present invention for use in the method of FIG. 1; and

FIG. 12 illustrates a system for estimating a stress level of a subject according to an embodiment of the present invention, the system implementing the method of FIG. 1.

While the invention is amenable to various modifications and alternative forms, specific embodiments are shown by way of example in the drawings and are described in detail. It should be understood, however, that the drawings and detailed description are not intended to limit the invention to the particular form disclosed. The intention is to cover all modifications, equivalents, and alternatives falling within the spirit and scope of the present invention as defined by the appended claims.

### DETAILED DESCRIPTION

In the following description, for the purposes of explanation, numerous specific details are set forth in order to provide a thorough understanding of the present invention. It will be apparent, however, that the present invention may be practiced without these specific details. In some instances, well-known structures and devices are shown in block diagram form in order to avoid unnecessary obscuring.

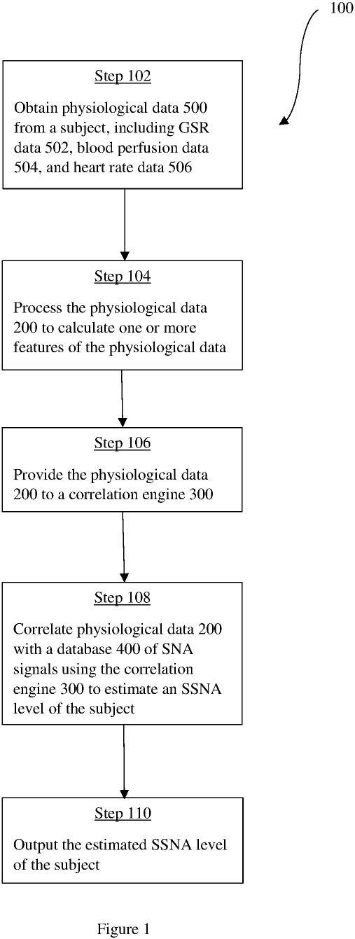

FIG. 1 is a flowchart illustrating a method 100 for estimating sympathetic arousal of a subject (not shown) according to an embodiment of the present invention. Sympathetic arousal of a subject can be used to estimate stress levels of the subject. The method 100 comprises the following steps:
Step 102 — obtaining physiological data 500 from the subject;
Step 104 — processing the physiological data 500 to calculate one or more features of the physiological data;
Step 106 — providing the physiological data 500 to a correlation engine 300;
Step 108 — correlating the physiological data 500 with a database 400 of skin sympathetic nervous activity (SNA) signals using the correlation engine 300 to estimate a SNA level of the subject; and
Step 110 — outputting the estimated SNA level of the subject.

For clarity reasons, generation of the correlation engine 300 and the database 400 will be described before describing the method 100 in more detail below.

### Generation of Correlation Engine 300 and Database 400

### Generating Correlation Engine 300 and Database 400 Using Raw SNA Signals Corresponding to SNA Signals

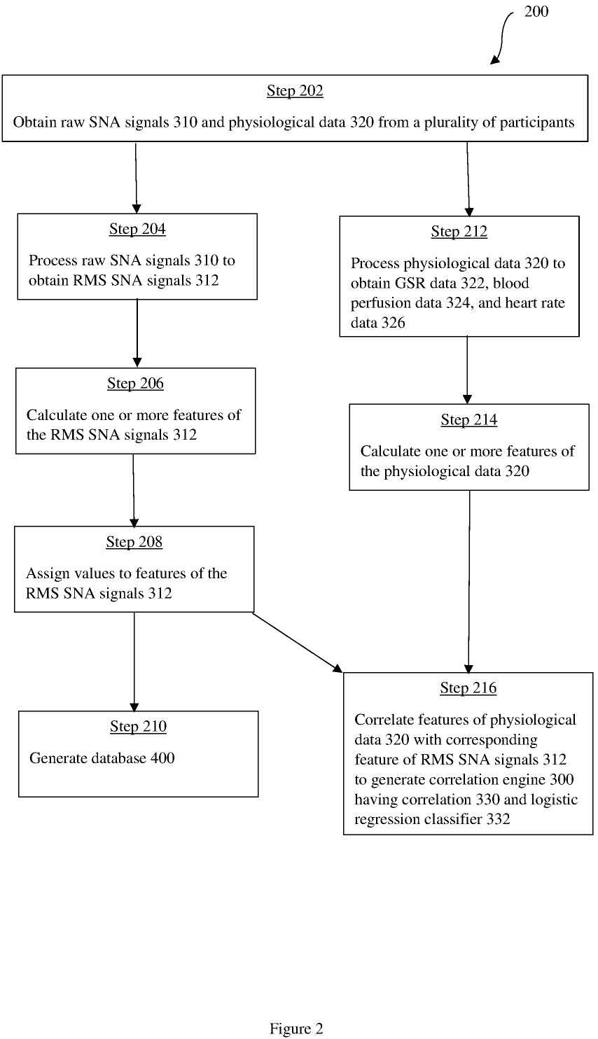

FIG. 2 is a flowchart illustrating a method 200 of generating the correlation engine 300 and the database 400 according to an embodiment of the present invention.

### Step 202 - Obtaining Raw SNA Signal Recordings and Physiological Data

At step 202, raw SNA signals 310 and corresponding physiological data 320 are obtained from a plurality of participants (not shown). For each participant, the raw SNA signals 310 and corresponding physiological data 320 may be obtained simultaneously.

The raw SNA signals 310 from each participant may be obtained using microneurography by inserting a high impedance intraneural probe (e.g. a tungsten electrode) percutaneously into a muscle fascicle of the participant to record, over an extended period, the participant’s skin sympathetic nervous activity (SSNA) and/or muscle sympathetic nervous activity (MSNA) at different stress levels. The signal provided by microneurological approach is regarded as the ‘gold standard’ for assessing stress level in a subject, ie. the most direct observation of the ‘fight or flight’ response. It will be appreciated that the raw SNA signals obtained using microneurography will be raw microneurographic (MNG) signals. It will also be appreciated that the raw SNA signals 310 may be obtained using any other suitable methods known in the art.

Obtaining the physiological data 320 may include initially obtaining skin conductivity data and/or photoplethysmography (PPG) from each participant. The skin conductivity data may be obtained using one or more skin conductivity sensors to measure the conductivity of each participant’s skin and the PPG data may be obtained using one or more PPG sensors. The one or more PPG sensors may be a single wavelength PPG sensor or a multiple wavelength PPG sensor. The Shimmer3 GSR + Unit from Shimmer™, Empatica Embrace, Empatica E4, Philips Health Band, and Microsoft Band are examples of sensor devices that may be used to obtain the skin conductivity data and/or PPG data from the participants.

When obtaining the raw SNA signals 310 and physiological data 320 from each participant, the participants may be exposed to different stressors using known methods in the art to record the participant’s raw SNA signals 310 and physiological data 320 at different stress levels. Accordingly, there will be multiple raw SNA signals 310 and physiological data 320 recorded from each participant as they are exposed to different stressors to record the reaction of the participant’s sympathetic nervous system at different stress levels. As will be appreciated, the raw SNA signals 310 represent sympathetic nervous activity signals of the respective participants.

It will be appreciated that the raw SNA signals 310 obtained from each participant at the different stress levels constitutes another form of physiological data of the participants. Accordingly, at step 202, physiological data is obtained from each participant at different stress levels using two different methods. The first method involves obtaining raw SNA signals 310 from each participant at different stress levels (e.g. via microneurography). The second method involves obtaining the physiological data 320 from each participant at different stress levels as described above. The raw SNA signals 310 thus form a first set of physiological data obtained from each participant at different stress levels, and the physiological data 320 form a second set of physiological data obtained from each participant at different stress levels.

### Step 204 - Processing Raw SNA Signal

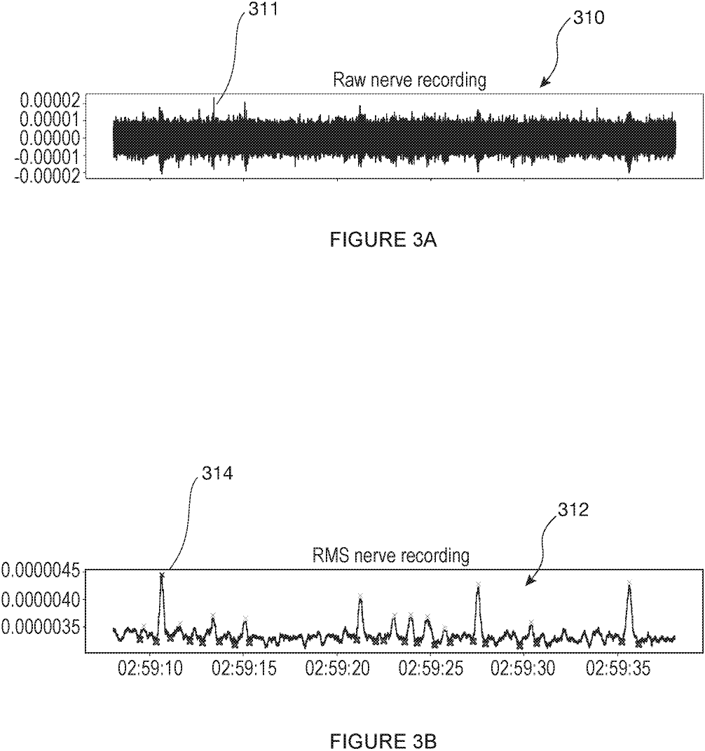

At step 204, the raw SNA signals 310 from each participant are processed. FIG. 3A is an example of such a baseline trace, i.e. the raw SNA signal over a period of time. The raw SNA signal illustrated in FIG. 3A is in the form of an MNG signal obtained via microneurography.

It can be seen that the raw SNA signal 310 contains spikes 311 and a significant amount of noise, the spikes 311 indicating activation of the sympathetic nervous system of the participant. Activation of the sympathetic nervous system is commonly referred to as a sympathetic nervous activity (SNA) burst.

Referring to FIG. 3B, the raw SNA signal 310 is processed using known methods in the art to produce an RMS SNA signal 312, in which the spikes 311 are transformed into SNA bursts 314. It will be appreciated that if the raw SNA signals 310 are MNG signals obtained using microneurography, then the RMS SNA 312 signals will be in the form of RMS MNG signals.

As illustrated in FIGS. 3A and 3B, the spikes 311 are integrated into the respective SNA bursts 314. Each SNA burst 314 indicating an activation of the sympathetic nervous system of the participant.

It will be appreciated that different methods can be employed for detecting true spikes 311 in the raw SNA signals 310 and, therefore, true SNA bursts 314 in the RMS SNA signals 312. Once the SNA bursts 314 have been detected, relevant features can be extracted from the RMS signal 312. One suitable approach is discussed below.

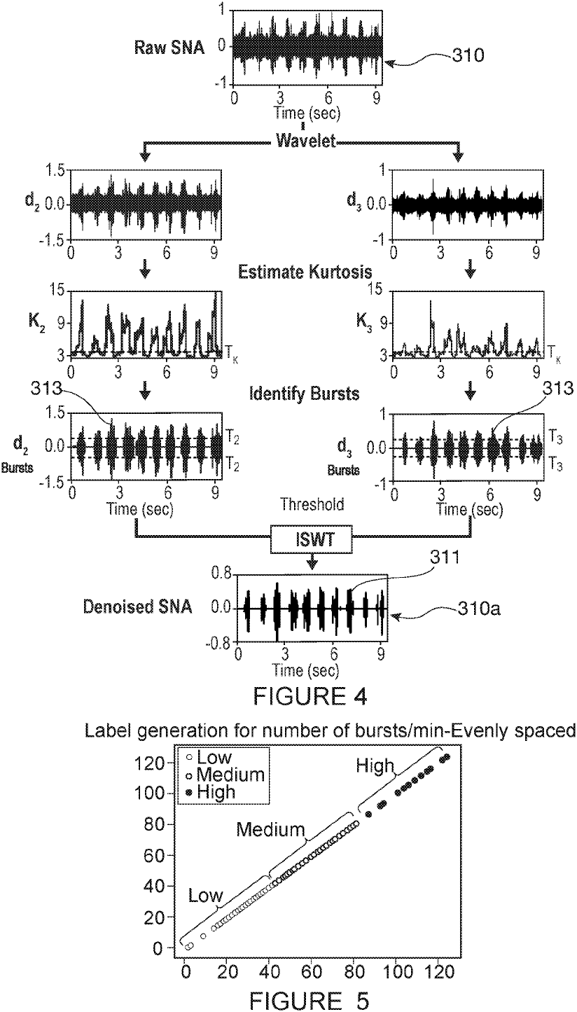

FIG. 4 illustrates an example method of processing the raw SNA signals 310 to distinguish spikes 311 in the raw SNA signals 310 from the background noise, to be used in identifying the SNA bursts 314 in the RMS SNA signals 312.

Shown at the top of FIG. 4 is an example raw SNA signal 310 that includes a large amount of noise. The raw SNA signal 310 is subjected to a wavelet transform step. Each subband undergoes a kurtosis estimation and a thresholding step to identify the SNA bursts. An inverse stationary wavelet transform is computed, which is used to estimate the locations of the SNA bursts 313.

The thresholding step discards any part of the SNA bursts 313 that do not meet a maximum and/or a minimum threshold value. The result of the thresholding step is a de-noised SNA signal 310a, shown at the bottom of FIG. 4. The raw SNA signal 310 is then processed using known methods in the art to produce the RMS SNA signal 312 having the SNA bursts 314 identified from the SNA bursts 313.

Although FIG. 4 illustrates a wavelet transform having two branches, it will be appreciated that a wavelet transform having a larger or smaller number of branches may also be used. Further, although the thresholding step has been described as removing noise from the SNA bursts 313 by discarding parts of the SNA bursts 313 that do not meet a maximum and/or minimum threshold value, it is also envisaged that other suitable methods known in the art that are capable of removing noise from the SNA bursts 313 to detect the spikes 311 may also be used.

### Step 206 - Calculating Features of RMS SNA Signals 312

Referring to FIG. 2, at step 206, each RMS SNA signal 312 from each participant is processed to calculate one or more features of the respective RMS SNA signal 312. Each RMS SNA signal 312 may be processed to calculate one or more of the following features of the RMS SNA signal 312:
the number of SNA bursts over a predetermined time period;
the number of SNA bursts per one hundred heart beats;
the total area under all of the SNA bursts;
the maximum amplitudes of the SNA bursts;
the median amplitude of the SNA bursts; and
the average duration of the SNA bursts.

Each of the above features may be calculated using known methods in the art. The above list is not exhaustive and each RMS SNA signal 312 may be processed to calculate one or more features not listed above.

### Step 208 - Assigning Values to Features of the RMS SNA Signal 312

At step 208, for each participant, the one or more features of each RMS SNA signal 312 calculated above in step 206 is assigned a value. Each value is indicative of an SNA level of the participant. The SNA level of the participant is an indicator of sympathetic arousal of the participant and, therefore, the stress level of the participant. The values may include:
A “low” value — indicating a low SSNA level;
A “medium” value — indicating a medium SSNA level; and
A “high” value — indicating a high SSNA level.

From the RMS SNA signals 312 obtained from each participant at different stress levels, it is possible to calculate a low, medium, and high SNA level for each feature calculated from the RMS SNA signals 312. For example:
a greater number of SNA bursts per predetermined time period or per one hundred heart beats may indicate a higher estimated SNA level and vice versa;
a larger total area under the SNA bursts may indicate a higher estimated SNA level and vice versa;
larger maximum amplitudes of the SNA bursts may indicate a higher estimated SNA level and vice versa;
a larger median amplitude of the SNA bursts may indicate a higher estimated SNA level and vice versa; and
longer average times of the SNA bursts may indicate a higher estimated SNA level and vice versa.

Although only three values have been described above, it will be appreciated that there could be more or less than three values if desired.

### Step 210 - Generating Database 400

At step 210, the RMS SNA signals 312, the one or more features calculated from the RMS SNA signals 312, and/or the values assigned to the features of the RMS SNA signals 312 are stored on the database 400. The raw SNA signals 310 may also be stored on the database 400.

As an example, the total number of SNA bursts per minute calculated from the RMS SNA signals 312 may be stored on the database 400. FIG. 5 is a graph illustrating the estimated SNA level for a given number of SNA bursts per minute. The graph of FIG. 5 has three distinct regions indicating low, medium, and high estimated SNA levels based on the number of SNA bursts per minute. It will be appreciated that other features of the RMS SNA signals 312 may also be stored on the database 400 in a similar manner.

It will be appreciated that the RMS SNA signals 312, the one or more features calculated from the RMS SNA signals 312, and the values assigned to the features of the RMS SNA signals 312 each represent sympathetic nervous activity. Accordingly, the database 400 represents sympathetic nervous activity that can be used to estimate a sympathetic nervous activity level of a subject.

### Step 212 - Processing Physiological Data

Referring to FIG. 2, at step 212, the physiological data 320 obtained from each participant at different stress levels at step 202 is processed. In particular, the skin conductivity data may be processed using known methods in the art to obtain galvanic skin response (GSR) data 322 and/or the PPG data may be processed to obtain skin blood perfusion data 324 and heart rate data 326. Accordingly, the physiological data 320 obtained from each participant at different stress levels includes at least one of GSR data 322, skin blood perfusion data 324, and heart rate data 326.

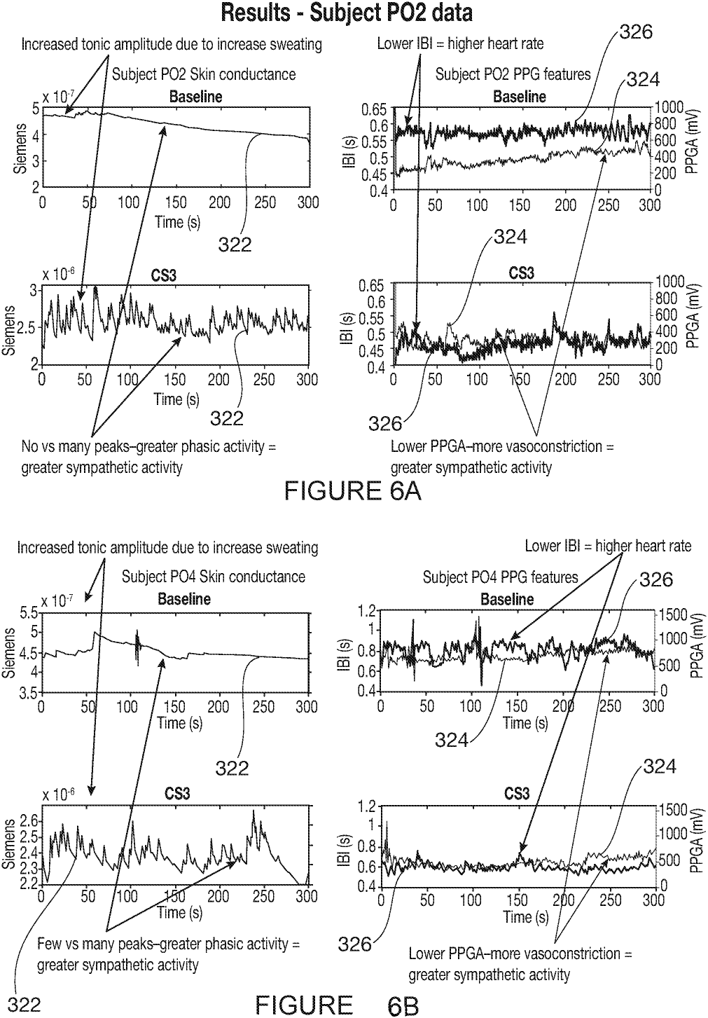

As discussed above, physiological data 320 is obtained from each participant at different stress levels. At each stress level, each of the GSR data 322, skin blood perfusion data 324, and heart rate data 326 comprise a signal having features that indicate changes in the skin conductivity, skin blood perfusion, and heart rate of the respective participant, respectively. FIGS. 6A and 6B show example GSR data 322, skin blood perfusion data 324, and heart rate data 326. From these figures, it can be seen that:
increased skin conductivity and greater phasic activity in the GSR data 322 may indicate greater sympathetic arousal of the participant;
increased vasoconstriction (i.e. decreased blood flow) in the skin blood perfusion data 324 may indicate greater sympathetic arousal of the participant; and
increased heart rate in the heart rate data 326 may indicate greater sympathetic arousal of the participant.

### Step 214 - Calculating One or More Features of Physiological Data

At step 214, the physiological data 320 obtained from each participant at the different stress levels is processed to calculate one or more features of the physiological data 320. In particular, the physiological data 320 is processed to calculate one or more features of at least one of the GSR data 322, skin blood perfusion data 324, and heart rate data 326. Accordingly, the one or more features of the physiological data 320 includes one or more features of the GSR data 322, and/or one or more features of the skin blood perfusion data 324, and/or one or more features of the heart rate data 326.

The GSR data 322 may be processed to calculate one or more of the following features of the GSR data 322:
GSR peak rise time sum: peak rise time = (time of occurrence of peak) — (time of point of onset);
GSR peak amplitude sum = (GSR value at peak) — (GSR value at point of onset);
GSR half-recovery sum = (time of occurrence of half amplitude) — (time of occurrence of peak);
GSR peak energy sum = 0.5 × peak amplitude × peak rise time;
GSR rise rate average = sum average of first derivative of points with first derivative >+ve threshold (0.025);
GSR decay rate average = sum average of first derivative of points with first derivative < a predetermined threshold;
GSR percentage decay = percentage of 1st derivative of points with first derivative < 0; and
GSR number of peak = number of peaks in the GSR data 322.

The skin blood perfusion data 324 may be processed to calculate one or more of the following features of the skin blood perfusion data 324:
PPG peak to peak amplitude (PPGA) = this feature corresponds to skin blood flow of each participant.

The heart rate data 326 may comprise heart rate variability (HRV) data, which may be processed to calculate one or more of the following features of the heart rate data 326:
SDNN = standard deviation of all NN intervals;
RMSSD = The square root of the mean of the sum of the squares of differences between adjacent NN intervals;
SDSD = standard deviation of differences between adjacent NN intervals;
NN50 count — 1 = number of pairs of adjacent NN intervals differing by more than 50 ms in the entire recording — only pairs in which the first interval is longer;
NN50 count — 2 = Number of pairs of adjacent NN intervals differing by more than 50 ms in the entire recording — only pairs in which the second interval is longer;
HRV triangular index = triangular index calculated from histogram; and
pNN50 = NN50 count divided by the total number of all NN intervals.

The above lists of features for the GSR data 322, the skin blood perfusion data 324, and the heart rate data 326 are not exhaustive. Accordingly, it will be appreciated that the GSR data 322, the skin blood perfusion data 324, and the heart rate data 326 may be processed to calculate one or more features not listed above.

### Step 216 - Correlating One or More Features of Physiological Data 320 With Features of RMS SNA Signals 312

At step 216, for each RMS SNA signal 312 obtained, the one or more features of the RMS SNA signal 312 are correlated with one or more features of the respective corresponding physiological data 320 to generate a correlation 330 between the RMS SNA signals 312 and the physiological data 320. For example:
one or more features of the GSR data 322, the skin blood perfusion data 324, and/or the heart rate data 326;
one or more features of the GSR data 322 and the skin blood perfusion data 324;
one or more features of the GSR data 322 and the heart rate data 326; and/or
one or more features of the skin blood perfusion data 324 and the heart rate data 326; and/or
one or more features of the heart rate data 326,
are correlated with one or more features of the respective corresponding RMS SNA signal 312 to generate a correlation 330. Accordingly, the correlation 330 is configured to correlate one or more features of physiological data with one or more features of the RMS SNA signals 312 stored on the database 400. The correlation 330 may be any type of correlation. For example, the correlation 330 may be a linear, exponential, Pearson’s, or any other type of correlation known in the art.

The correlation 330 forms the basis of the correlation engine 300, which also comprises a machine learning algorithm in the form of a logistic regression classifier 332. As the skilled reader will understand, logistic regression is a statistical method for assigning an input feature vector to one of multiple target categories.

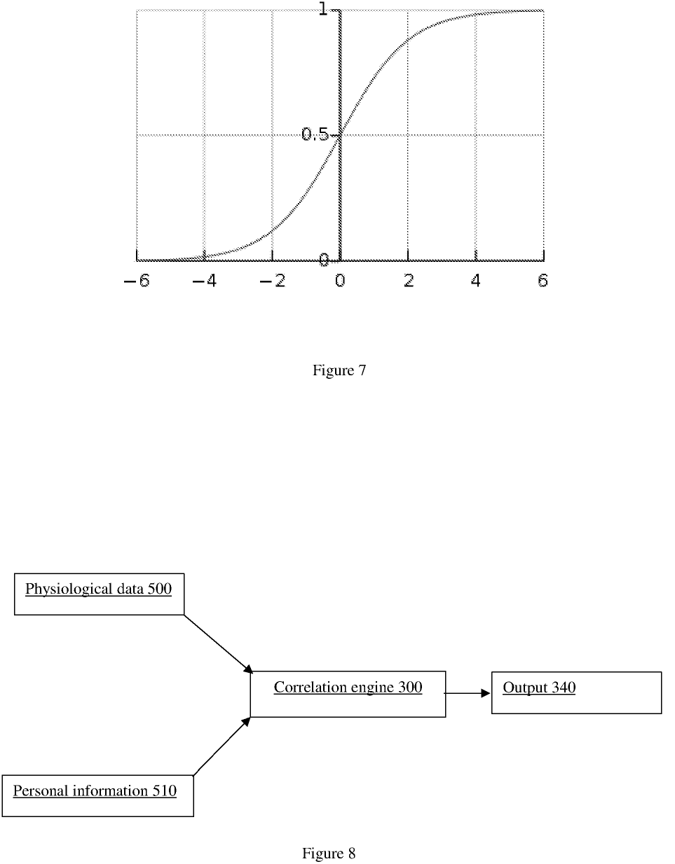

Logistic regression classifier 332 is a supervised machine learning algorithm that is trained to predict SNA levels from physiological data. Logistic regression uses a logistic function which is typically a sigmoid function (see FIG. 7), the equation being:

$$S \left(x\right) = \frac{1}{1 + {e}^{- x}}$$

This function ensures that S(x) is always between 0 and 1:

$$S \left(x\right) = \frac{1}{1 + {e}^{- x}} = \frac{{e}^{x}}{{e}^{x} + 1}$$

According to an embodiment, in binomial (binary classification) logistic regression, the linear combination of the inputs is passed through the logistic function to create the classifier output:

$$\hat{y} = {β}_{0} + {β}_{1} {x}_{1} + ⋯ + {β}_{n} {x}_{n}$$

$$p = \frac{1}{1 + {e}^{- \hat{y}}}$$

The variable p is the probability that the input (e.g. the estimated number of SNA bursts per minute) belongs to the specified category (i.e. low, medium, or high). This is calculated using weights βi and physiological features xᵢ.

The training process calculates the optimal values of weights βi for the logistic regression classifier. To find the optimal weights, a set of training data with known categories is used. The optimization process searches the β₁ - βₙ space for the set of weights that would result in minimum classification error over the training set. The training process for the logistic regression classifier 332 thus comprises generating correlation 330.

Referring to FIG. 8, the correlation engine 300 is configured to receive physiological data 500 from a subject (not shown) and, on the basis of the correlation 330, estimate one or more features of the RMS SNA signals 312 from the physiological data 500. The logistic classifier 332 is configured, based on the training process described above, to classify the estimated one or more features of the RMS SNA signals 312 as low, medium, or high, which will be an estimate of the subjects SNA level. According to an embodiment, the classifications of the estimated one or more features of the RMS SNA signals 312 may be averaged to generate an average classification (e.g. low, medium, or high) for the estimated one or more features of the RMS SNA signals 312, which will provide an estimate of the subject’s SNA level. According to another embodiment, a weighted average may be calculated for the classifications of the estimated one or more features of the RMS SNA signals 312 where particular features of the RMS SNA signals 312 are assigned a greater weight, which will provide an estimate of the subject’s SNA level. It is also envisaged that combinations of the averaging and weighted averaging methods described above may be used to estimate the SNA level of a subject.

As an example, the correlation engine 300 may be configured to receive physiological data 500 from a subject and, based on the correlation 330, estimate the number of SNA bursts per minute (i.e. one feature of the RMS SNA signals) from the physiological data 500. In this example, the logistic classifier 332 is configured, based on the training process described above, to classify the estimated number of SNA bursts per minute as low, medium, or high, which will be an estimate of the subjects SNA level.

The correlation engine 300 is therefore able to correlate one or more features of the physiological data 500 with one or more features of the RMS SNA signals 312 to estimate the SNA level of a subject. The inventors have found that correlating the physiological data 500 with the RMS SNA signals 312 in accordance with the present invention provides a significantly more reliable estimate of the subject’s SNA level than otherwise would be possible.

Although the above has been described with reference to the feature of SNA bursts per minute of the RMS SNA signals 312, it will be appreciated that one or more other features of the RMS SNA signals 312 may be used.

It will also be appreciated that obtaining the raw SNA signals 310 and the corresponding physiological data 320 simultaneously from each participant at step 202 may allow for the RMS SNA signals 312 to be more accurately correlated with the respective corresponding physiological data 320 at step 214, which may, therefore, improve the accuracy of the correlation engine 300.

Estimating an SNA level of a subject may be affected by other factors personal to the subject. Referring to FIG. 8, the correlation engine 300 may therefore also be configured to receive additional information 510 concerning the subject and to correlate the physiological data 500 with one or more features of the RMS SNA signals 312 stored on the database 400 in accordance with the additional information 510, in order to estimate the SNA level of the subject.

The additional information 510 may include the subject’s age, demographic data, weight, chronic stress level, etc. The additional information 510 may be used as another factor when calculating the weight βi for the logistic regression classifier 332 discussed above. Accordingly, correlation engine 300 may be configured to use the additional information 510 with regard to calculating the weights βi in the logistic regression classifier 332 and, therefore, influence the estimated SNA level of the subject.

### Generating Correlation Engine 300 and Database 400 Using ICG Signals

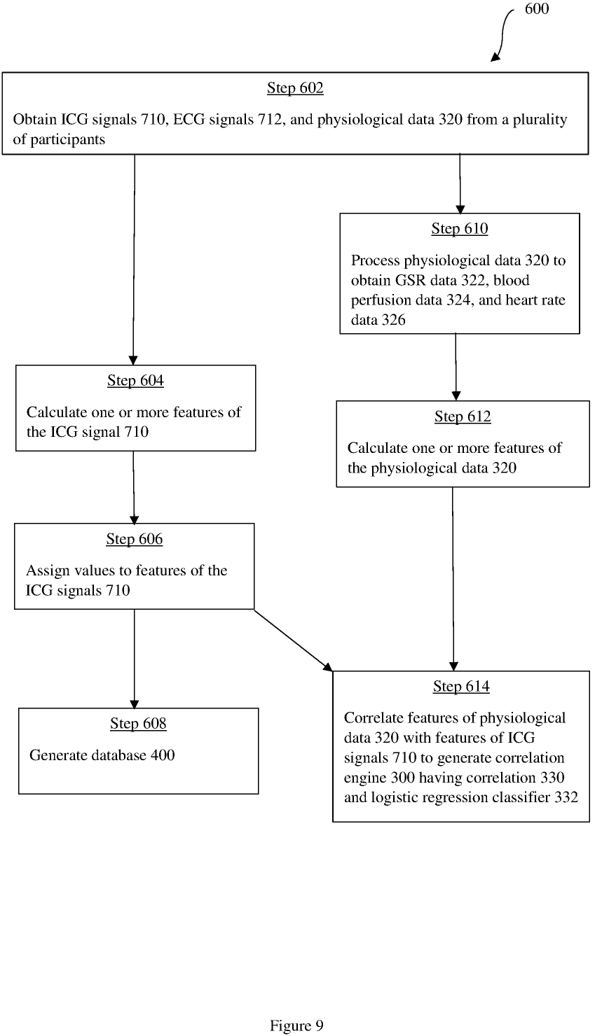

FIG. 9 is a flowchart illustrating an alternative method 600 of generating the correlation engine 300 and the database 400 according to a further embodiment of the present invention.

### Step 602 - Obtaining ICG and ECG Signal Recordings and Physiological Data

At step 602, impedance-cardiography (ICG) signals 710, electrocardiogram (ECG) signals 712, and corresponding physiological data 320 are obtained from a plurality of participants (not shown). For each participant, the ICG signals 710, ECG signals 712, and corresponding physiological data 320 may be obtained simultaneously. The physiological data 320 may be obtained as described above at step 202 for method 200. The ICG signals 710 and ECG signals 712 may be obtained using any suitable methods known in the art.

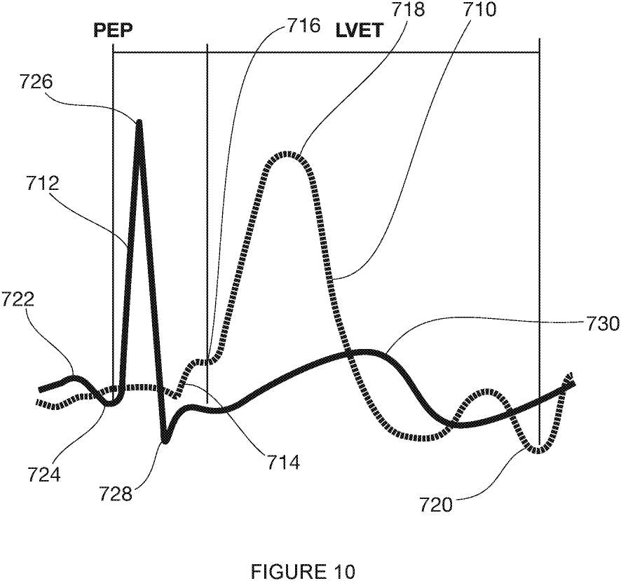

FIG. 10 is an example of an ICG signal 710 and ECG signal 712 obtained from a participant. The ICG signal 710 has an A point 714, a B point 716, a C point 718, and an X point 720. The ECG signal 712 has a P wave 722, a Q wave 724, an R wave 726, an S wave 728, and a T wave 730. The time difference between the Q wave 724 of the ECG signal 712 and the B point 716 of the ICG signal 710 is know is the pre-ejection period (PEP). The time difference between the B wave 716 and the X wave 720 of the ICG signal 710 is known as the left ventricular ejection time (LVET). LVET is commonly referred to as the ventricular ejection time.

When obtaining the ICG signals 710, ECG signals 712, and physiological signal 320 from each participant, each of the participants may be exposed to different stressors using known methods in the art to record the participant’s ICG signals 710, ECG signals 712 and physiological data 320 at different stress levels. Accordingly, there will be multiple ICG signals 710, ECG signals 712 and physiological data 320 recorded from each participant as they are exposed to different stressors. The ICG signals 710, ECG signals 712, and physiological data 320 may be used to determine the reaction of each participant’s sympathetic nervous system at different stress levels. It will therefore be appreciated that the ICG signals 710, the ECG signals 712, and the physiological data 320 represent sympathetic nervous activity of the respective participants.

It will be appreciated that the ICG signals 710 and ECG signals 712 obtained from each participant at the different stress levels constitute other forms of physiological data of the participants. Accordingly, at step 602, physiological data is obtained from each participant at different stress levels using two different methods. The first method being obtaining ICG signals 710 and ECG signals 712 from each participant at different stress levels. The second method being obtaining the physiological data 320 from each participant at different stress levels as described above. The ICG signals 710 and ECG signals 712 form a first set of physiological data obtained from each participant at different stress levels. The physiological data 320 form a second set of physiological data obtained from each participant at different stress levels.

Step 604 - calculating features of ICG signals 810Referring to FIG. 9, at step 604, each of the ICG signals 710 and ECG signals 712 from each participant is processed to calculate one or more features of the respective ICG signal 710. Each ICG signal 710 may be processed to calculate one or more of the following features:
Stroke volume (mL) of the heart;
Heart rate in beats per minute (BPM);
Cardiac output of the heart (litres/minute);
Ventricular ejection time of the heart (seconds); and
Change in the pre-ejection period (PEP) of the heart (seconds).

The above list of features for the ICG signals 710 are not exhaustive and other features may be calculated from the ICG signals 710. The above list of features may be calculated using any suitable methods know in the art.

### Step 606 - Assigning Values to Features of ICG Signals 710

At step 606, for each participant, the one or more features of each ICG signal 710 calculated above in step 604 is assigned a value similar to step 208 described above with respect to method 200

From the ICG signals 710 obtained from each participant at different stress levels, it is possible to calculate a low, medium, and high SNA level for each feature calculated from the ICG signals 710. For example:
Variations in the stroke volume may indicate a change in the SNA level ;
A higher heart rate may indicate a higher SNA level and vice versa;
Variations in the cardiac output may indicate a change in the SNA level;
Variations in the ventricular ejection time may indicate a change in the SNA level; and
A decrease in the PEP from baseline may indicate a higher SNA level and vice versa.

For example, during sympathetic arousal (i.e. an increase in the SNA level of the participant), the PEP of a participant will reduce from baseline conditions, if the posture of the participant is controlled. Accordingly, the change in the PEP of a participant may be calculated using the following formula:

$$P E {P}_{c h a n g e} = P E {P}_{a r o u s a l} - P E {P}_{b a s e l i n e}$$

Accordingly, the change in the PEP of a participant may be used to estimate the SNA level of the participant.

### Step 608 - Generating Database 400

At step 608, similar to step 210 for method 200 described above, the ICG signals 710, the one or more features calculated from the ICG signals 710, and the values assigned to the features of the ICG signals 710 are stored on the database 400.

As an example, the change in PEP calculated from the ICG signals 710 may be stored on the database 400. Similar to what was described with respect to FIG. 5, different ranges of the change in PEP may be used to indicate low, medium, and high estimated SNA levels. It will be appreciated that other features of the ICG signals 710 may also be stored on the data base 400 in a similar manner.

It will be appreciated that the ICG signals 710, the one or more features calculated from the ICG signals 710, and the values assigned to the features of the ICG signals 710 each represent sympathetic nervous activity. Accordingly, the database 400 represents sympathetic nervous activity that can be used to estimate a sympathetic nervous activity level of a subject.

### Step 610 - Processing Physiological Data

At step 610, the physiological data 320 obtained from each participant at different stress levels at step 602 is processed in the same manner described above at step 212 for method 200.

### Step 612 - Calculating One or More Features of Physiological Data

At step 612, the physiological data 320 obtained from each participant at the different stress levels is processed to calculate one or more features of the physiological data 320 using the same methods described above at step 214 for method 200.

### Step 614 - Correlating One or More Features of Physiological Data 320 With One or More Features of ICG Signals 710

At step 614, for each ICG signal 710 obtained, the one or more features of the ICG signals 710 are correlated with one or more features of the respective physiological data 320 to generate a correlation 330 between the ICG signals 710 and the physiological data 320. For example:
one or more features of the GSR data 322, the skin blood perfusion data 324, and/or the heart rate data 326;
one or more features of the GSR data 322 and the skin blood perfusion data 324;
one or more features of the GSR data 322 and the heart rate data 326; and/or
one or more features of the skin blood perfusion data 324 and the heart rate data 326; and/or
one or more features of the heart rate data 326,
are correlated with one or more features of the respective corresponding ICG signal 710 to generate a correlation 330. Accordingly, the correlation 330 is configured to correlate one or more features of physiological data 320 with one or more features of the ICG signals 710 stored on the database 400. The correlation 330 may be any type of correlation. For example, the correlation 330 may be a linear, exponential, Pearson’s, or any other type of correlation known in the art.

The correlation 330 forms the basis of the correlation engine 300, which also comprises a machine learning algorithm, in the form of the logistic regression classifier 332 described above at step 210 of method 200.

Referring to FIG. 8, the correlation engine 300 is configured to receive physiological data 500 from a subject (not shown) and, on the basis of the correlation 330, estimate one or more features of the ICG signals 710 from the physiological data 500. The logistic classifier 332 is configured, based on the training process described above, to classify the estimated one or more features of the ICG signals 710 as low, medium, or high, which will be an estimate of the subject’s SNA level. According to an embodiment, the classifications of the estimated one or more features of the ICG signals 710 may be averaged to generate an average classification (e.g. low, medium, or high) for the estimated one or more features of the ICG signals 710, which will provide an estimate of the subject’s SNA level. According to another embodiment, a weighted average may be calculated for the classifications of the estimated one or more features of the ICG signals 710 where particular features of the ICG signals 710 are assigned a greater weight, which will provide an estimate of the subject’s SNA level. It is also envisaged that combinations of the averaging and weighted averaging methods described above may be used to estimate the SNA level of a subject.

As an example, the correlation engine 300 may be configured to receive physiological data 500 from a subject and, based on the correlation 330, estimate the change in PEP (i.e. one feature of the ICG signals 710) from the physiological data 500. In this example, the logistic classifier 332 is configured, based on the training process described above, to classify the change in PEP as low, medium, or high, which will be an estimate of the subject’s SNA level. The correlation engine 300 is therefore able to correlate one or more features of the physiological data 500 with one or more features of the ICG signals 710 to estimate the SNA level of a subject.

Although the above has been described with reference to the feature of PEP of the ICG signals 710, it will be appreciated that one or more features of the ICG signals 710 may be used.

It will be appreciated that obtaining the ICG signals 710, the ECG signals 712, and the corresponding physiological data 320 simultaneously from each participant at step 602 may allow for the ICG signals 710 to be more accurately correlated with the respective corresponding physiological data 320 at step 612, which may, therefore, improve the accuracy of the correlation engine 300.

Further, similar to that described above with respect to method 200, estimating an SNA level of a subject may be affected by other factors personal to the subject. Referring to FIG. 8, the correlation engine 300 may therefore be configured to receive additional information 510 concerning the subject and to correlate the physiological data 500 with one or more features of the ICG signals 710 stored on the database 400 in accordance with the additional information 510 in order to estimate the SNA levels of the subject.

The information that may be included with the additional information 510 and how the additional information 510 may be used by the correlation engine 300 are described above with respect to step 216 of method 200.

### Generating Correlation Engine 300 and Database 400 Using Raw SNA Signals and ICG Signals

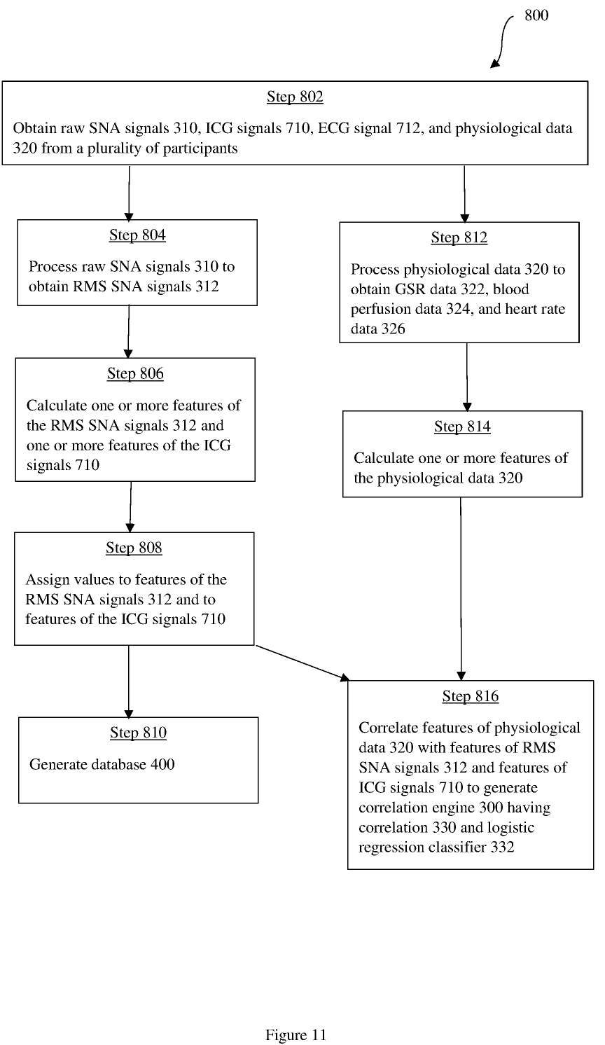

FIG. 11 is a flowchart illustrating an alternative method 800 of generating the correlation engine 300 and the database 400 according to a further embodiment of the present invention.

### Step 802 - Obtaining Raw SNA Signal Recordings, ICG Signal Recording, ECG Signal Recordings, and Physiological Data

At step 802, raw SNA signals 310, ICG signals 710, ECG signals 712 and corresponding physiological data 320 are obtained from a plurality of participants (not shown). For each participant, the raw SNA signals 310, ICG signals 710, ECG signals 712 and corresponding physiological data 320 may be obtained simultaneously. The raw SNA signals 310 may be obtained as described above at step 202 for method 200. The ICG signals 710 and ECG signals 712 may be obtained as described above at step 602 for method 600. The physiological data 320 may be obtained as described above at step 202 for method 200.

It will be appreciated that the raw SNA signals 310, the ICG signals 710 and the ECG signals 712 obtained from each participant at the different stress levels constitute other forms of physiological data of the participants. Accordingly, at step 802, physiological data is obtained from each participant at different stress levels using three different methods. The first method involves obtaining raw SNA signals 310 from each participant at different stress levels (e.g. via microneurography). The second method involves obtaining ICG signals 710 and ECG signals 712 from each participant at different stress levels. The third method involes obtaining the physiological data 320 from each participant at different stress levels as described above. The raw SNA signals 310, the ICG signals 710 and the ECG signals 712 form a first set of physiological data obtained from each participant at different stress levels. The physiological data 320 form a second set of physiological data obtained from each participant at different stress levels.

### Step 804 - Processing Raw SNA Signal

At step 804, the raw SNA signals 310 from each participant are processed as described above at step 204 for method 200.

### Step 806 - Calculating Features of RMS SNA Signals 312 and ICG Signals 710

At step 806, each RMS SNA signal 312 from each participant is processed to calculate one or more features of the respective RMS SNA signal 312 as described above at step 206 for method 200. Further, each of the ICG signals 710 and ECG signals 712 from each participant is processed to calculate one or more features of the respective ICG signal 710 as described above at step 604 for method 600.

### Step 808 - Assigning Values to Features of the RMS SNA Signal 312 and ICG Signals 710

At step 808, for each participant, the one or more features of each RMS SNA signal 312 calculated above in step 806 is assigned a value as described above at step 208 for method 200. Further, for each participant, the one or more features of each ICG signal 710 calculated above in step 806 is assigned a value as described above at step 606.

### Step 810 - Generating Database 400

At step 810, the RMS SNA signals 312, the one or more features calculated from the RMS SNA signals 312, and/or the values assigned to the features of the RMS SNA signals 312 are stored on the database 400. The raw SNA signals 310 may also be stored on the database 400. Further, the ICG signals 710, the one or more features calculated from the ICG signals 710 and the values assigned to the features of the ICG signals 710 are also stored on the database 400.

As described above, the RMS SNA signals 312, the one or more features calculated from the RMS SNA signals 312, the values assigned to the features of the RMS SNA signals 312, the ICG signals 710, the one or more features calculated from the ICG signals 710 and the values assigned to the features of the ICG signals 710 each represent sympathetic nervous activity. In other words, while some of these measures provides a direct representation of SNA, others represent a recognised correlate with sympathetic nervous activity, in effect being used as proxy measures of SNA signals. Accordingly, the database 400 represents sympathetic nervous activity that can be used to estimate a sympathetic nervous activity level of a subject.

### Step 812 - Processing Physiological Data

At step 812, the physiological data 320 obtained from each participant at different stress levels at step 802 is processed as described above at step 212 for method 200.

### Step 814 - Calculating One or More Features of Physiological Data

At step 814, the physiological data 320 obtained from each participant at the different stress levels is processed to calculate one or more features of the physiological data 320 as described above at step 214 for method 200.

### Step 816 - Correlating One or More Features of Physiological Data 320 With Features of RMS SNA Signals 312 and ICG Signals 710

At step 816, for each RMS SNA signal 312 and ICG signal 710 obtained, the one or more features of the RMS SNA signal 312 and the one or more features of the ICG signals 710 are correlated with one or more features of the respective corresponding physiological data 320 to generate a correlation 330 between the RMS SNA signals 312 and the physiological data 320, and between the ICG signal 710 and the physiological data 320. For example:
one or more features of the GSR data 322, the skin blood perfusion data 324 and/or the heart rate data 326;
one or more features of the GSR data 322 and the skin blood perfusion data 324;
one or more features of the GSR data 322 and the heart rate data 326; and/or
one or more features of the skin blood perfusion data 324 and the heart rate data 326; and/or
one or more features of the heart rate data 326,
are correlated with one or more features of the respective corresponding RMS SNA signal 312 and with one or more features of the respective corresponding ICG signal 710 to generate a correlation 330. Accordingly, the correlation 330 is configured to correlate one or more features of physiological data with one or more features of the RMS SNA signals 312 and with one or more features of the ICG signals 710 stored on database 400. The correlation 330 may be any type of correlation. For example, the correlation 330 may be a linear, exponential, Pearson’s, or any other type of correlation known in the art.

The correlation 330 forms the basis of the correlation engine 300, which also comprises a machine learning algorithm, in the form of the logistic regression classifier 332 described above at step 210 of method 200.

Referring to FIG. 8, the correlation engine 300 is configured to receive physiological data 500 from a subject (not shown) and, on the basis of the correlation 330, estimate one or more features of the RMS SNA signals 312 and/or one or more features of the ICG signals 710 from the physiological data 500. The logistic classifier 332 is configured, based on the training process described above, to classify the estimated one or more features of the RMS SNA signal 312 and the one or more features of the ICG signals 710 as low, medium, or high, which will provide an estimate of the subject’s SNA level. According to an embodiment, the classifications of the estimated one or more features of the RMS SNA signals 312 may be averaged to generate an average classification (e.g. low, medium, or high) for the estimated one or more features of the RMS SNA signals 312, the classifications of the estimated one or more features of the ICG signals 710 may be averaged to generate an average classification (e.g. low, medium, or high) for the estimated one or more features of the ICG signals, and the average classification for the estimated one or more features of the RMS SNA and ICG signals may be averaged to provide an estimate of the subject’s SNA level. According to another embodiment, a weighted average may be calculated for the classifications of the estimated one or more features of the RMS SNA signals 312 where particular features of the RMS SNA signals 312 are assigned a greater weight, a weighted average may be calculated for the classifications of the estimated one or more features of the ICG signals 710 where particular features of the ICG signals 710 are assigned a greater weight, and a weighted average may be calculated for the weighted average of the estimated one or more features of the RMS SNA and ICG signals to provide an estimate of the subject’s SNA level. It is also envisaged that combinations of the averaging and weighted averaging methods described above may be used to estimate the SNA level of a subject.

As an example, the correlation engine 300 may be configured to receive physiological data 500 from a subject and, based on the correlation 330, estimate the number of bursts per minute (i.e. one feature of the RMS SNA signals 312) and/or the change in PEP (i.e. one feature of the ICG signals 710) from the physiological data 500. In this example, the logistic classifier 332 is configured, based on the training process described above, to classify the number of bursts per minute and the change in PEP as low, medium, or high, which will provide an estimate of the subject’s SNA level. The correlation engine 300 is therefore able to correlate one or more features of the physiological data 500 with one or more features of the RMS SNA signals 312 and one or more features of the ICG signals 710 to estimate the SNA level of a subject.

Although the above has been described with reference to the feature of the number of bursts per minute of the RMS SNA signals 312 and the change in PEP of the ICG signals 710, it will be appreciated that one or more features of the RMS SNA signals 312 and one or more features of the ICG signals 710 may be used.

It will be appreciated that obtaining the raw SNA signals 310, the ICG signals 710, the ECG signals 712, and the corresponding physiological data 320 simultaneously from each participant at step 802 may allow for the RMS SNA signals 312 and the ICG signals 710 to be more accurately correlated with the respective corresponding physiological data 320 at step 814, which may therefore improve the accuracy of the correlation engine 300.

Further, similar to that described above with respect to method 200, estimating an SNA level of a subject may be affected by other factors personal to the subject. Referring to FIG. 8, the correlation engine 300 may therefore be configured to receive additional information 510 concerning the subject and to correlate the physiological data 500 with one or more features of the RMS SNA signal 312 and/or one or more features of the ICG signals 710 stored on the database 400 in accordance with the additional information 510 in order to estimate the SNA levels of the subject.

The information that may be included with the additional information 510 and how the additional information 510 may be used by the correlation engine 300 are described above with respect to step 216 of method 200.

Although estimating the SNA level of a subject has been described above as using a logistic regression classifier, it will be appreciated that other suitable machine learning algorithms known in the art that operate in a similar manner to the logistic regression classifier 332 may be used instead or in addition. Still further, although the correlation engine 300 has been described as having a machine learning algorithm in the form of a logistic regression classifier, it is envisaged that any other suitable machine learning algorithm known in the art that is capable of performing a similar function to the logistic regression classifier 332 may be used.

### Method 100

Referring to FIG. 1, the method 100 for estimating a stress level of a subject is described below.

### Step 102 - Obtaining Physiological Data 500 From the Subject

At step 102, physiological data 500 is obtained from the subject (not shown). The physiological data 500 includes at least one of GSR data 502, skin blood perfusion data 504, and heart rate data 506 of the subject.

Obtaining the physiological data 500 may include initially obtaining skin conductivity data and/or PPG data from the subject. The skin conductivity data may be obtained using one or more skin conductivity sensors to measure the conductivity of the subject’s skin and the PPG data may be obtained using one or more PPG sensors. The skin conductivity data is processed using known methods in the art to obtain the GSR data and/or the PPG data is processed using known methods in the art to obtain the skin blood perfusion data and the heart rate data.

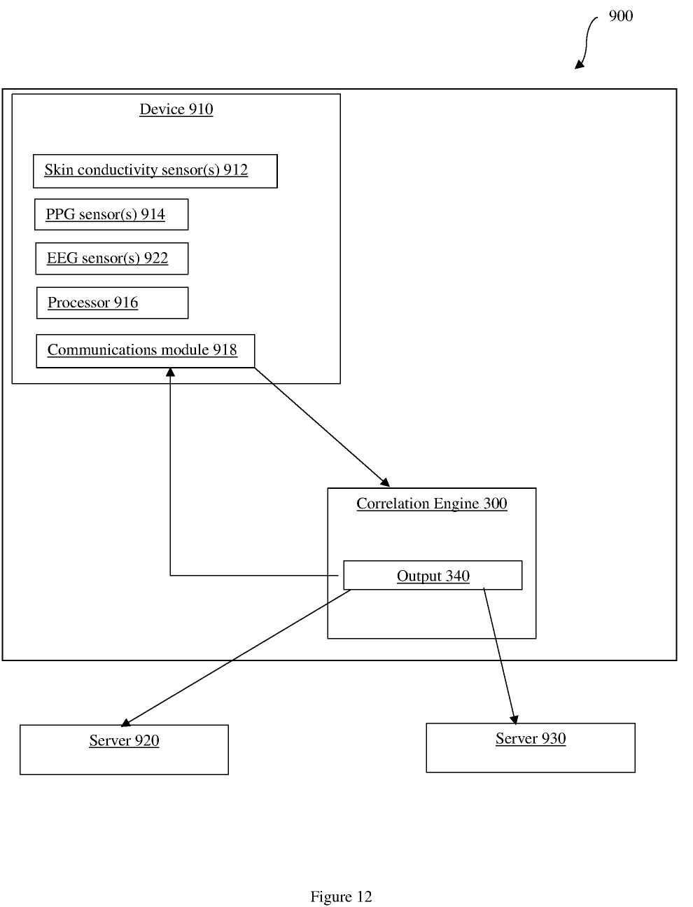

As an example, the one or more skin conductivity sensors and the one or more PPG sensors may be integrated into a wearable device (e.g. see FIG. 12) that contacts the subject’s skin. For example, the wearable device may be a smart watch that is worn on the wrist of a subject.

### Step 104 - Processing Physiological Data 500 to Calculate Features of the Physiological Data 500

At step 104, the physiological data 500 is processed to calculate one or more features of the physiological data 500. In particular, the physiological data 500 is processed to calculate one or more features of at least one of the GSR data 502, skin blood perfusion data 504, and heart rate data 506. The physiological data 500 may be processed to calculate one or more of the features listed above in respect to step 214 of the method 200. It will be appreciated that those lists of features are not exhaustive and that the physiological data 500 may be processed to calculate one or more features not listed.

### Step 106 - Providing Physiological Data 500 to Correlation Engine 300

At step 106, the one or more features of the physiological data 500 calculated in step 104 are provided as an input to the correlation engine 300 described above.

### Step 108 - Estimating Sympathetic Nervous Activity Level of Subject

At step 108, and as described above, the correlation engine 300 is configured to correlate the one or more features of the physiological data 500 with one or more features of the RMS SNA signals 312 and/or one or more features of the ICG signals 710 to estimate an SNA level of the subject. In particular, the correlation engine 300 is configured to correlate:
one or more features of the GSR data 502, the skin blood perfusion data 504, and/or the heart rate data 506;
one or more features of the GSR data 502 and the skin blood perfusion data 504;
one or more features of the GSR data 502 and the heart rate data 506;
one or more features of the skin blood perfusion data 504 and the heart rate data 506; and/or
one or more features of the heart rate data 506,
with one or more features of the RMS SNA signals 312 and/or one or more features of the ICG signals 710 to estimate an SNA level of the subject.

Similar to that described above with respect to steps 216, 614, and 816 of the respective methods 200, 600, and 800, the correlation engine 300 may also be configured to receive personal information (see FIG. 8) from the subject and correlate the physiological data 500 with one or more features of the RMS SNA signals 312 and/or one or more features of the ICG signals 710 stored on the database 400 based on the personal information 510 to estimate the SNA level of the subject.

### Step 110 - Outputting Estimated Sympathetic Nervous Activity Level of Subject

At step 110, the correlation engine 300 is configured to generate an output 340 of the estimated SNA level of the subject. The output 340 may be in the form of a report that is sent to the subject and/or to a health clinician so that the health clinician can manage and record the subject’s mental health and intervene if necessary.

The method of the invention therefore involves using various of the features extracted from the subject’s physiological data as inputs to the correlation engine, which produces as an output a score predicting the subject’s stress level.

### Correlating Physiological Data With EEG Signals

According to another embodiment, at steps 202, 602, and 802, electroencephalogram (EEG) signals (not shown) may also obtained from each participant at different stress levels. For each participant, the EEG signals may be obtained simultaneously with the physiological data 320 and raw SNA signals 310, ICG signals 710, and/or ECG signals 712.

At steps 206, 602, and 802, the EEG signals from each participant are processed to calculate one or more features of the EEG signals. For example, features that may be calculated from the EEG signals include, but are not limited to:
Entropy;
Absolute power;
Relative power;
Maximum power; and/or
Centre of frequency for the following band:
   Delta: 0.5 - 3.5 Hz frequency range of the EEG signal;
   Theta: 4.0 - 7.5 Hz frequency range of the EEG signal;
   Alpha: 8.0 - 13.0 Hz frequency range of the EEG signal;
   Beta: 14.0 - 32 Hz frequency range of the EEG signal; and/or
   SEn: sample entropy.

At steps 208, 606, and 808, for each participant, the one or more features of each EEG signal are assigned a value indicative of the participant’s sympathetic arousal.

At steps 210, 608, and 810, the one or more features calculated from the EEG signals and the values assigned to the features of the EEG signals are stored on the database 400.

At steps 216, 614, and 816, for each EEG signal obtained, the features calculated from the respective corresponding physiological data 320 are also correlated with the one or more features of the EEG signals to generate the correlation 330. Accordingly, the correlation 330 is configured to correlate features of physiological data 320 with one or more features of:
the RMS SNA signals 312 and the EEG signals stored on database 400 (in the case in which database 400 is generated using only raw SNA signals 310);
the ICG signals 710 and the EEG signals stored on database 400 (in the case in which database 400 is generated using only raw SNA signals 310); and
the RMS SNA signals, the ICG signals 710, and/or the EEG signals stored on database 400 (in the case in which database 400 is generated using both raw SNA signals 310 and ICG signals 710).

For these embodiments, at step 108, one or more features of the physiological data 500 obtained from the subject are correlated with one or more features of:
the EEG signals and the RMS SNA signals 312 sored on database 400 (in the case in which database 400 is generated using only raw SNA signals 310);
the EEG signal and the ICG signals 710 stored on database 400 (in the case in which database 400 is generated using only ICG signals 710 and ECG signals 712); or
the EEG signals, the RMS SNA signal 312, and/or the ICG signals 710 stored on database 400 (in the case in which database 400 is generated using both raw SNA signals 310 and ICG signals 710),
to estimate an SNA level of the subject.

### Correlating SNA Signals And/or ICG Signals With EEG Data

According to another embodiment, at steps 202, 602, and 802, the physiological data 320 obtained from each participant at different stress levels may also include EEG data (not shown). For each participant, the EEG data may be obtained simultaneously with the raw SNA signal 310 and/or ICG signal 710.

At steps 212, 610, and 812, the EEG data from each participant is processed to calculate one or more features of the EEG data. For example, the EEG data may be processed to calculate one or more of the features listed above.

At steps 216, 614, and 816, for the EEG data obtained from each participant at different stress levels, the one or more features of the EEG data are correlated with one or more features of:
the respective corresponding RMS SNA signals 312 to generate the correlation 330 (in the case in which database 400 is generated with only raw SNA signal 310);
the respective corresponding ICG signals 710 to generate the correlation 330 (in the case in which database 400 is generated with only ICG signals 710 and ECG signals 712); and
the respective corresponding RMS SNA signals 312 and ICG signals 712 to generate the correlation 330 (in the case in which database 400 is generated with both raw SNA signals 310 and ICG signals 710).

Accordingly, the correlation 330 is configured to correlate one or more features of the EEG data with one or more features of the RMS SNA signals 312 and/or ICG signals 710.

For these embodiments, at step 108, the physiological data 500 obtained from the subject may include EEG data and one or more features of the EEG data are correlated with one or more features of:
the RMS SNA signals 312 (in the case in which database 400 is generated with only raw SNA signal 310);
the ICG signals 710 (in the case in which database 400 is generated with only ICG signals 710 and ECG signals 712); and
the RMS SNA signals 312 and/or ICG signals 710 (in the case in which database 400 is generated with both raw SNA signals 310 and ICG signals 710),
to estimate an SNA level of the subject.

### System 900

FIG. 12 is a block diagram of a system 900 for estimating sympathetic arousal of a subject (not shown) according to an embodiment of the present invention. The system 900 implements the method 100 described above for estimating the SNA level of the subject. The system 900 comprises a device 910, and the correlation engine 300.

The device 910 may be a wearable device that contacts the subject’s skin. For example, the wearable device may be a smart watch that is worn on the wrist of a subject. The device 910 has sensors 911 that are configured to obtain physiological data 500 from the subject. The sensors 911 include one or more skin conductivity sensors 912 and/or one or more PPG sensors 914. The device 910 also has a processor 916 and a communications module 918.

The skin conductivity sensors 912 may be electrical contacts that are configured to obtain skin conductivity data from the subject by measuring the conductivity of the subject’s skin. It is also envisaged that the skin conductivity sensors 912 may be any other suitable sensors known in the art that are capable of measuring the conductivity of the subject’s skin.

The PPG sensors 914 are configured to obtain PPG data from the subject. The PPG sensors 914 may be a single wavelength PPG sensor or a multiple wavelength PPG sensor. However, it is also envisaged that the device 910 may instead use alternative sensors known in the art that are capable of measuring/obtaining skin blood perfusion data and heart rate data. Alternatively, the device 910 may have a sensor to measure/obtain the skin blood perfusion data and another separate sensor to measure/obtain the heart rate data.

The device 900 may also include EEG sensors 922 configured to obtain EEG data from the subject.

The processor 916 is configured to process the skin conductivity data to obtain GSR data 502 of the subject and/or to process the PPG data to obtain skin blood perfusion data 504 and/or heart rate data 506 of the subject. The skin conductivity data is processed using known methods in the art to obtain the GSR data 502 and/or the PPG data is processed using known methods in the art to obtain the skin blood perfusion data 504 and/or the heart rate data 506. Accordingly, the physiological data 500 obtained from the subject includes at least one of the GSR data 502, the skin blood perfusion data 504, and the heart rate data 506.

The processor 916 is also configured to process the physiological data 500 to calculate one or more features of the physiological data 500. In particular, the processor 916 is configured to process the physiological data 500 to calculate one or more features of at least one of the GSR data 502, skin blood perfusion data 504, and heart rate data 506. Accordingly, the one or more features of the physiological data 500 includes one or more features of the GSR data 502, and/or one or more features of the skin blood perfusion data 504, and/or one or more features of the heart rate data 506. The one or more features calculated from each the GSR data 502, skin blood perfusion data 504, and/or heart rate data 506 may include the features listed and described above with respect to step 102 of the method 100.

The communications module 918 is configured to transmit the one or more features of the physiological data 500 to the correlation engine 300. As described above, the correlation engine 300 correlates the one or more features of the physiological data 500 with one or more features of the RMS SNA signals 312 and/or one or more features of the ICG signals 710 to estimate, and generate an output 340 of, the SNA level of the subject.

The correlation engine 300 may be configured to report the estimated SNA level of the subject to the subject by sending the output 340 in the form of a report to the device 910 and/or to a server 920 that can be accessed by the subject through a computing device (e.g. a computer, mobile phone, tablet computer, etc.) to view the output 340. The device 910 may be further configured to send the output 340 in the form of a report to a server 930 that can be accessed by a health clinician through a computing device (e.g. a computer, mobile phone, tablet computer, etc.) to view the output 340. This may allow the health clinician to manage and record the subject’s mental health remotely. Further, as the system 900 may allow for real-time reporting of the subject’s stress levels in the ambulatory environment, the health clinician may be able to intervene sooner and provide improved health care to the subject compared to the prior art methods of monitoring and reporting stress levels.

Although FIG. 12 illustrates the correlation engine 300 and database 400 as being separate from the device 910, it is envisaged that the correlation engine 300 and the database 400 may be stored in memory (not shown) on the device 910. In this case, the processor 914 may be further configured to initiate the correlation engine 300, input the physiological data 500 into the correlation engine 300, and access the database 400 to estimate the SNA level of the subject.

In another embodiment of the system 900, the correlation engine 300 and the database 400 may be hosted on a server (not shown) that is configured to communicate with the communication module 918 of the device 910. In this embodiment, the physiological data 500 processed by the processor 916 is sent by the communication module 918 to the server. The server is configured to initiate the correlation engine, input the physiological data 500 into the correlation engine 300, and access the database 400 to estimate the SNA level of the subject. The server may be further configured to transmit the output 340 of the estimated SNA level of the subject to the device 910 via the communications module 618 and/or to transmit the output 340 to the server 920 and/or server 930.

In another embodiment of the system 900, the processor 916, the correlation engine 300, and the database 400 may be hosted on a server (not shown) that is configured to communicate with the communication module 918 of the device 910. In this embodiment, the physiological data 500 obtained from the one or more skin conductivity sensors 912 and one or more PPG sensors 914 is sent by the communication module 918 to the server. The server is configured to utilize the processor 916 to process the physiological data to obtain the GSR data, the skin blood perfusion data, and the heart rate data. The server is also configured to initiate the correlation engine 300, input the physiological data 500 processed by the processor 916 into the correlation engine 300, and access the database 400 to estimate the SNA level of the subject. The server may be further configured to transmit the output 340 of the estimated SNA level of the subject to the device 910 via the communications module 618 and/or to transmit the output 340 to the server 920 and/or server 930.

It will be appreciated that the system 900 may not include the server 930. In this embodiment, the correlation engine 300 may be configured to transmit the output 340 to the server 920, which may be accessed by both the subject and health clinician through a computing device (e.g. a computer, mobile phone, tablet computer, etc.) to view the output 340.

In light of the above, it will be appreciated that the method 100 may allow a stress level of a subject to be estimated relatively quickly and accurately using a combination of physiological data obtained from the subject. It will also be appreciated that the system 900 implementing the method 100 may allow a stress level of a subject to be measured relatively quickly and accurately in the ambulatory environment. Accordingly, accurate reporting of the subject’s stress levels may be reported to a health clinician who may be able to more effectively record and manage a subject’s stress levels and possibly intervene sooner using the method 100 and system 900, thereby providing improved health care to the subject and potentially improving the subject’s mental health.

### Validation

Test were conducted to validate the accuracy of the method 100 and system 900 for estimating the stress level of a subject. In this experiment, the accuracy of the method 100 and system 900 was tested by correlating different combinations of physiological data 500 obtained from the subject with different features of the RMS signals 312 stored on the database 400. The different combinations of physiological data 500 obtained from the subject are listed in the first column of each of the Tables 1 - 3 below. The accuracy of these different combination is as a percentage value in each of Tables 1 - 3 below. Further:
in Table1, the features of the RMS SNA signals 312 stored on the database 400 were assigned one of three values (e.g. low, medium, or high);
in Table 2, the features of the RMS SNA signals 312 stored on the database 400 were assigned one of four values (e.g. low, low-medium, medium- high, or high); and
in Table 3, the features of the RMS SNA signals 312 stored on the database 400 were assigned one of five values (e.g. low, low-medium, medium, medium-high, or high).

TABLE 1

| Feature combination | Bursts/min | Bursts/100 hbs | Total area | Median peak | Max peak | Avg duration | Mean accuracy |
|---|---|---|---|---|---|---|---|
| GSR + PPGA + HR + HRV | 89.58% | 85.94% | 94.03% | 85.42% | 95.29% | 94.32% | 90.76% |
| GSR + HR + HRV | 87.94% | 83.85% | 83.80% | 82.73% | 94.59% | 90.45% | 87.23% |
| HR + HRV | 87.54% | 79.06% | 83.95% | 85.64% | 92.93% | 92.42% | 86.92% |
| PPGA + HR + HRV | 86.73% | 80.21% | 83.72% | 81.17% | 92.93% | 92.42% | 86.20% |
| GSR + HR | 86.24% | 84.29% | 78.96% | 81.17% | 93.71% | 89.92% | 85.82% |
| GSR + PPGA + HR | 85.04% | 82.98% | 78.96% | 80.82% | 93.71% | 89.92% | 85.24% |
| GSR + PPGA | 86.37% | 81.14% | 77.82% | 80.03% | 94.93% | 89.92% | 85.04% |
| GSR | 85.83% | 82.73% | 80.37% | 80.63% | 91.01% | 88.92% | 84.91% |
| PPGA + HR | 83.24% | 84.54% | 73.86% | 88.75% | 92.64% | 85.61% | 84.77% |
| HR | 70.63% | 76.19% | 68.94% | 87.50% | 90.49% | 81.44% | 79.20% |

TABLE 2

| Feature combination | Bursts/min | Bursts/100 hbs | Total area | Median peak | Max peak | Avg duration | Mean accuracy |
|---|---|---|---|---|---|---|---|
| GSR + PPGA + HR + HRV | 94.00% | 92.92% | 88.19% | 88.65% | 94.99% | 90.02% | 91.46% |
| GSR + HR + HRV | 92.35% | 92.35% | 86.61% | 88.65% | 94.48% | 90.45% | 90.81% |
| GSR + HR | 90.60% | 90.60% | 86.63% | 86.07% | 93.98% | 93.33% | 90.20% |
| GSR + PPGA + HR | 90.60% | 90.60% | 84.78% | 86.07% | 93.98% | 93.48% | 89.92% |
| GSR + PPGA | 89.64% | 89.90% | 87.30% | 87.09% | 93.52% | 91.16% | 89.77% |
| GSR | 91.66% | 91.66% | 86.19% | 80.54% | 90.91% | 91.27% | 88.71% |
| HR + HRV | 90.73% | 88.64% | 81.37% | 87.09% | 93.77% | 89.94% | 88.59% |
| PPGA + HR + HRV | 88.87% | 89.54% | 81.37% | 85.52% | 93.77% | 89.31% | 88.06% |
| PPGA + HR | 87.50% | 87.50% | 74.64% | 89.59% | 90.82% | 81.94% | 85.33% |
| HR | 86.61% | 86.61% | 72.86% | 88.34% | 90.66% | 79.41% | 84.08% |

TABLE 3

| Feature combination | Bursts/min | Bursts/100 hbs | Total area | Median peak | Max peak | Avg duration | Mean accuracy |
|---|---|---|---|---|---|---|---|
| GSR + PPGA + HR + HRV | 89.04% | 90.54% | 90.10% | 86.99% | 94.67% | 90.28% | 90.27% |
| GSR + HR + HRV | 87.45% | 88.61% | 89.03% | 85.56% | 94.26% | 89.86% | 89.13% |
| GSR + HR | 84.08% | 86.16% | 92.10% | 84.87% | 94.99% | 92.08% | 89.05% |
| PPGA + HR + HRV | 89.42% | 91.26% | 85.01% | 84.54% | 94.73% | 86.44% | 88.57% |
| GSR + PPGA + HR | 84.08% | 84.87% | 89.83% | 84.87% | 94.99% | 92.08% | 88.45% |
| GSR + PPGA | 84.38% | 84.87% | 90.06% | 83.65% | 94.67% | 90.66% | 88.05% |
| GSR | 86.10% | 86.10% | 87.10% | 82.46% | 90.91% | 89.02% | 86.95% |
| HR + HRV | 85.67% | 85.67% | 83.93% | 84.83% | 93.77% | 85.42% | 86.55% |
| PPGA + HR | 77.53% | 82.34% | 79.51% | 89.59% | 92.54% | 79.93% | 83.57% |
| HR | 76.15% | 78.32% | 78.93% | 88.34% | 93.21% | 76.82% | 81.96% |

From Tables 1 - 3, it can be seen that:
increasing the number of physiological inputs to be correlated with RMS SNA signal 312 increases the accuracy;
increasing the number of values assigned to the features of the RMS SNS signals 312 stored on the database 400 does not affect the accuracy significantly; and
the feature of the RMS SNA signals 312 that provided the most accurate results is the maximum amplitude of the SNA burst in the RMS SNA signals 312.

As used herein the terms “include” and “comprise” (and variations of those terms, such as “including”, “includes”, “comprising”, “comprises”, “comprised” and the like) are intended to be inclusive and are not intended to exclude further features, components, integers or steps.

Various features of the disclosure have been described using flowcharts. The functionality/processing of a given flowchart operation could potentially be performed in various different ways and by various different systems or applications. Furthermore, it may be possible for a given flowchart operation to be divided into multiple operations and/or multiple flowchart operations to be combined into a single operation. Furthermore, in some instances the order of the steps may be able to be changed without departing from the scope of the present disclosure.

It will be understood that the embodiments disclosed and defined in this specification extends to all alternative combinations of two or more of the individual features mentioned or evident from the text or drawings. All of these different combinations constitute various alternative aspects of the embodiments.

1. A method for estimating sympathetic arousal of a subject, the method comprising the steps of:
obtaining physiological data from the subject, the physiological data including at least one of galvanic skin response data, skin blood perfusion data, and heart rate data of the subject;
processing the physiological data to determine one or more features of the physiological data;
providing the one or more features of the physiological data to a correlation engine, the correlation engine configured to correlate the one or more features of the physiological data with a database representing sympathetic nervous activity signals to estimate a sympathetic nervous activity level of the subject; and
generating an output of the estimated sympathetic nervous activity level of the subject.

2. The method of claim 1, wherein correlation engine is configured to correlate the one or more features of the physiological data with one or more features of the sympathetic nervous activity signals.

3. The method of claim 2, wherein the one or more features of the physiological data comprises one or more features of at least one of the galvanic skin response data, the skin blood perfusion data, and the heart rate data.

4. The method of claim 3, wherein the correlation engine is configured to correlate one or more features of the sympathetic nervous activity signals with a combination of features selected from the one or more features of the galvanic skin response data, skin blood perfusion data, and heart rate data.

5. The method of any one of claims 2 to 4, wherein each feature of the sympathetic nervous activity signals is assigned a value indicative of a sympathetic nervous activity level.

6. The method of any one of the preceding claims, wherein obtaining the physiological data comprises obtaining skin resistance data and photoplethysmography data from the subject.

7. The method of claim 6, wherein the skin resistance data is processed to obtain the galvanic skin response data and the photoplethysmography data is processed to obtain the skin blood perfusion data and the heart rate data.

8. The method of any one of the preceding claims, wherein the correlation engine is a machine learning algorithm configured to correlate the features of the physiological data with the sympathetic nervous activity signals stored on the database.

9. The method of claim 8, wherein the machine learning algorithm comprises a regression classifier configured to estimate the sympathetic nervous activity level of the subject.

10. The method of any one of the preceding claims, further comprising the step of storing the physiological data and the estimated skin sympathetic nervous activity level of the subject on a server.

11. The method of any one of the preceding claims, further comprising the step of sending the physiological data and/or the estimated sympathetic nervous activity level of the subject to the subject and/or to a health clinician.

12. The method of any one of the preceding claims, further comprising the step of sending the physiological data and/or the one or more features of the physiological data to a server hosting the correlation engine.

13. A method of developing a correlation engine for estimating sympathetic arousal of a subject, the method comprising the steps of:
generating a database representing sympathetic nervous activity by recording and processing representations of sympathetic nervous activity obtained from a plurality of participants;
obtaining physiological data from each participant, the physiological data of each participant including at least one of galvanic skin response data, skin blood perfusion data, and heart rate data of the subject;
processing the physiological data obtained from each participant to calculate one or more features of the physiological data; and
correlating each representation of sympathetic nervous activity obtained from each participant with the one or more features of the physiological data obtained from the respective participant to develop the correlation engine,
wherein the correlation engine is configured to be used for correlating one or more features of physiological data obtained from the subject with the representations of sympathetic nervous activity in the database in order to output an estimated sympathetic nervous activity level of the subject.

14. The method of claim 13, further comprising the step of:
processing each representation of sympathetic nervous activity obtained from each participant to calculate one or more features of each representation of sympathetic nervous activity obtained from each participant, wherein:
the correlation step comprises correlating the one or more features of each representation of sympathetic nervous activity of each participant with the one or more features of the physiological data of the respective participant to develop the correlation engine; and
the correlation engine is configured to correlate one or more features of the physiological data obtained from the subject with the one or more features of the representations of sympathetic nervous activity in the database to estimate the sympathetic nervous activity level of the subject.

15. The method of claim 14, further comprising the step of assigning a value to each feature of each representation of sympathetic nervous activity obtained from each participant, wherein each value is indicative of an estimated sympathetic nervous activity level.

16. The method of any one of claims 13 to 15, wherein processing the physiological data of each participant to calculate one or more features of the physiological data comprises processing at least one of the galvanic skin response data, skin blood perfusion data, and heart rate data to calculate one or more features of at least one of the galvanic skin response data, skin blood perfusion data, and heart rate data.

17. The method of claim 16, wherein the correlation step involves correlating a combination of features selected from the one or more features of the galvanic skin response data, the skin blood perfusion data, and the heart rate data of each participant with the one or more features of the representations of sympathetic nervous activity of the respective participant.

18. The method of any one of claims 14 to 17, wherein the representations of sympathetic nervous activity of each participant comprise one or more sympathetic nervous activity bursts, each sympathetic nervous activity burst indicating when the sympathetic nervous system of the participant was activated.

19. The method of claim 18, wherein calculating the one or more features of each representation of sympathetic nervous activity of each participant comprises calculating one or more of:
a number of times the sympathetic nervous system of the participant was activated over a predetermined time period;
a number of times the sympathetic nervous system of the participant was activated per one hundred heart beats;
a total area underneath the sympathetic nervous activity burst(s) ;
a maximum amplitude of the sympathetic nervous activity burst(s);
a median amplitude of the sympathetic nervous activity burst(s); and
an average duration of the sympathetic nervous activity burst(s).

20. The method of any one of claims 13 to 19, wherein the representations of sympathetic nervous activity of each participant include signals obtained using microneurography.

21. The method of any one of claims 13 to 20, wherein the representations of sympathetic nervous activity of each participant include signals obtained using a high impedance intraneural probe inserted percutaneously into each participant.

22. The method of any one of claims 13 to 21, wherein the representations of sympathetic nervous activity of each participant include skin sympathetic nervous activity signals or muscle sympathetic nervous activity signals obtained from each participant.

23. The method of any one of claims 13 to 22, wherein the representations of sympathetic nervous activity of each participant include impedance cardiography (ICG) signals obtained from each participant at different stress levels.

24. The method of claim 23, wherein each ICG signal is processed to calculate a change in a pre-ejection period (PEP) of each participant, the change in the PEP indicating when the sympathetic nervous system of the participant was activated.

25. The method of any one of claims 13 to 24, wherein obtaining the physiological data of each participant comprises:
obtaining skin conductance data and photoplethysmography data from the participant;
processing the skin conductance data to obtain the galvanic skin response data; and
processing the photoplethysmography data to obtain the skin blood perfusion data and the heart rate data.

26. The method of any one of claims 13 to 25, wherein the correlation engine is a machine learning algorithm comprising a regression classifier to estimate the sympathetic nervous activity level of the subject.

27. A system of estimating sympathetic arousal of a subject, the system comprising:
a device configured to obtain physiological data of the subject, the physiological data including at least one of galvanic skin response data, photoplethysmography data, and heart rate data of the subject;
a processor to process the physiological data to calculate one or more features of the physiological data; and
a correlation engine configured to correlate the one or more features of the physiological data with a database representing sympathetic nervous activity signals to estimate a sympathetic nervous activity level of the subject and output an estimate of a sympathetic nervous activity level of the subject.

28. The system of claim 27, wherein the correlation engine is configured to correlate the one or more features of the physiological data with one or more features of the sympathetic nervous activity signals.

29. The system of claim 28, wherein the one or more features of the physiological data comprises one or more features of at least one of the galvanic skin response data, the skin blood perfusion data, and the heart rate data.

30. The method of claim 29, wherein the correlation engine is configured to correlate one or more features of the sympathetic nervous activity signals with a combination of features selected from the one or more features of the galvanic skin response data, skin blood perfusion data, and heart rate data.

31. The system of any one of claims 28 to 30, wherein each feature of each sympathetic nervous activity signal is assigned a value indicative of a sympathetic nervous activity level.

32. The system of any one of claims 27 to 31, wherein:
the device comprises:
electrical contacts to obtain skin resistance data from the subject: and
one or more photoplethysmography sensors to obtain photoplethysmography data from the subject, and
the processor is configured to process the skin resistance data to obtain the galvanic skin response data and process the photoplethysmography data to obtain the skin blood perfusion data and the heart rate data.

33. The system of any one of claims 28 to 32, wherein the device comprises the processor, the database, and correlation engine.

34. The system of any one of claims 28 to 32, further comprising a server, wherein:
the processor, the database, and the correlation engine are hosted on the server; and
the device is configured to communicate with the server to send the physiological data to the server.

35. The system of any one of claims 27 to 32, further comprising a server, wherein:
the database and the correlation engine are hosted on the server; and
the device is configured to communicate with the server to send the physiological data processed by the processor to the server.

36. The system of any one of claims 27 to 35, wherein the correlation engine is a machine learning algorithm comprising a logistic regression classifier to estimate the sympathetic nervous activity level of the subject.

37. The system of any one of claims 27 to 36, wherein the device is a wearable device.
